(19) United States
(12) Patent Application Publication
(10) Pub. No.: US 2002/0128179 A1
(43) Pub. Date: Sep. 12, 2002

(54) Shaped microparticles for pulmonary drug delivery

(76) Inventors: William C. Tacon, Westerville, OH (US); Anthony A. Boiarski, Hilliard, OH (US); Carl F. Grove, Upper Arlington, OH (US); Richard S. Brody, Worthington, OH (US)

Correspondence Address: Patricia A. Coburn, Battelle Pulmonary Therapeutics, Inc., Suite 100, 1801 Watermark Drive, Columbus, OH, 43215, US

(21) Appl. No.: 10/020,464

(22) Filed: Nov. 30, 2001

Related U.S. Application Data
(60) Provisional application No. 60/250,717, filed on Dec. 1, 2000.

(51) Int. Cl. A61K 48/00; A61L 9/04; A61K 9/14; A61K 39/395

(52) U.S. Cl. 514/2; 424/46; 514/44; 424/130.1

(57) ABSTRACT

Microparticles for use in the pulmonary delivery of a therapeutic material, comprising a polymer matrix, which is prefabricated to have a particular geometric shape including that of a disc cube, rectangle or snowflake. Additionally, these microparticles may include a winged structure to enhance the aerodynamic characteristics of said microparticle. Microfabrication methods for making these microparticles are provided.

### CROSS-REFERENCE TO RELATED APPLICATION

The present application claims the benefit of U.S. Provisional Application No. 60/250,717 (Attorney Docket No. 12905P), filed Dec. 1, 2000.

### BACKGROUND OF INVENTION

This invention relates to prefabricated, shaped microparticles for use in pulmonary drug delivery by means of inhaled aerosols.

Preferred features of a pulmonary drug delivery platform include methods of delivering therapeutic molecules to specific regions of the patient's pulmonary system, particularly the lungs. For example, asthma compounds are targeted to the upper airways, whereas the deep regions of the lung (alveoli) are targeted for systemic delivery of molecules into the blood of a patient. Such region-specific targeting of the patient's pulmonary system can be achieved through the use of shaped microparticles falling within a precise size range. For example, small spherical particles in the 1 to 2 micron range are suitable for reaching the deep lung, whereas particles in the 3 to 5 micron range are useful for penetrating the upper airways.

Shaped microparticles have been designed, manufactured and used for drug delivery. For example, U.S. Pat. No. 6,107,102 issued to Ferrari (2000) discloses non-spherical microfabricated microdevices with a diameter in the range of 0.1 to 3 microns, for intravenous drug delivery of therapeutics.

Another preferred feature of a pulmonary delivery platform is sustained release of a particular material, such as a drug, from a microparticle over at least a 2 to 48 hour period. The sustained release approach to drug delivery often reduces the number of separate drug administrations that must be given to a patient. Sustained release microparticle systems are well characterized in the prior art.

Although there are currently a variety of drug delivery technologies in development for delivering therapeutic materials to the lung by means of inhalation, none of these technologies provides the combination of (i) microparticles of precisely controlled size; (ii) microparticles with shapes that enhance the aerodynamic characteristics of the microparticles; and (iii) microparticles which provide sustained release of a drug over a defined period of time. Microparticles having this combination of characteristics effectively enable the targeting of specific regions of the lung, such as the upper airways and the deep alveolar regions, for drug delivery. Thus, there is a need for a pulmonary drug delivery system that provides these features.

### SUMMARY OF INVENTION

These and other deficiencies of the prior art are overcome by the present invention, which provides microparticles for use in the pulmonary delivery of a therapeutic material. These microparticles comprise a polymer matrix, which is prefabricated to have a particular geometric shape including that of a disc cube, rectangle or snowflake.

The microparticles of the present invention have geometric diameter (width) of about 1 to 100 microns, a thickness of about 1 to 10 microns, and are intend to encapsulate or bind a variety of therapeutic materials including drugs, enzymes, hormones, proteins, antibodies, vitamins, peptides, polypeptides, nucleic acids, oligonucleotides, vaccines, cells, antigens, allergens, viruses. These microparticles are intended to be aerosolizable by dry powder nebulizers, liquid nebulizers, and electrostatic sprayers.

One embodiment of the polymer matrix of the present invention includes at least one biodegradable or biocompatible polymer such as polylactide or polyphosphazene. Another embodiment includes at least one additional polymer for enhancing the degradation characteristics of the polymer matrix.

Methods for making the shaped microparticles of this invention include microfabrication techniques such as cutting the microparticles from sheets of polymer by photolithography, microstamping the microparticles from sheets of polymer, or casting the microparticles in molds. Layered microparticles are also provided.

### BRIEF DESCRIPTION OF THE DRAWINGS

FIG. 1 depicts the disc-like, cubical, and rectangular embodiments of the microparticles of the present invention.

FIG. 2 depicts the snowflake embodiment of the microparticles of the present invention.

### DETAILED DESCRIPTION OF THE INVENTION

The present invention utilizes small “shaped” microparticles as an effective way to administer therapeutic materials to the pulmonary system of a patient. Drugs or other materials are bound to, incorporated in, or encapsulated by these shaped microparticles. Target-specific drug delivery, and controlled release of materials from microparticles, are important features of the present invention.

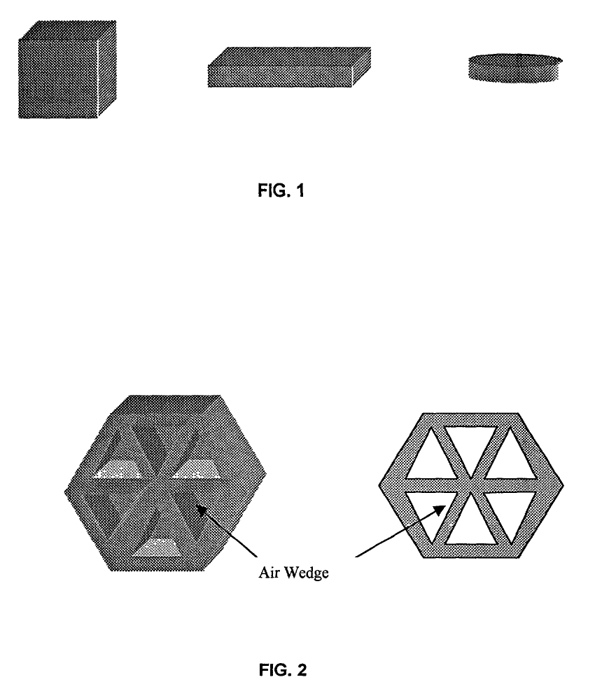

The preferred embodiment of the present invention provides shaped, i.e., non-spherical, microfabricated microparticles for pulmonary drug delivery. As shown in FIG. 1, preferred shapes include discs, cubes, or rectangles. Another embodiment is the “snowflake” design depicted in FIG. 2. Presumably, the geometry of the “snowflake” embodiment confers certain aerodynamic advantages to the microparticles, including improved flight characteristics, and reduced aerodynamic diameter due to the “air wedges” built into the particle. Various embodiments of the “snowflake” particle shown in FIG. 3, include a microparticle wherein the air wedges are either open holes, or are enclosed pockets which serve as microreservoirs for drugs, enzyme inhibitors, or other materials.

For effective delivery to all regions of the patient's pulmonary system, particles in the range of about 1 to 100 microns in width, with a thickness in the range of about 1 to 10 microns are preferred. Advantageously, microparticles with such large geometric diameters (i.e., width) and small thicknesses are retained in the lungs for the prolonged period required for sustained release of drugs. This effect is observed because microparticles with these preferred physical characteristics are more difficult for the patient's cells to endocytose than are smaller particles. Microparticles with large geometric diameters and small thicknesses also provide a large surface to volume ratio that can be advantageous for therapies utilizing surface bound drugs or ligands.

In general, the preferred methodology of the present invention utilizes a BioMEMS (Biological Micro Electro Mechanical Systems) microfabrication approach to generate “shaped” microparticles of a specific size or sizes. Preferred approaches include cutting microparticles from sheets of polymer by photolithography, microstamping microparticles from sheets of polymer, or by casting such particles in molds having the preferred shape.

Appropriate microfabrication techniques are disclosed in U.S. Pat. No. 6,107,102 which includes non-spherical microfabricated microdevices with a diameter in the range of 0.1 to 3 microns, for intravenous drug delivery of therapeutics. The specification of U.S. Pat. No. 6,107,102 is hereby incorporated by reference in its entirety. Similarly, WO 00/41740 describes materials and methods for the manufacture of asymmetrical microfabricated particles with a diameter in the range of 100 microns to 1 mm, for the oral delivery of proteins and peptides. The specification of WO 00/41740 is hereby incorporated by reference in its entirety.

By employing the processes described above, particles of precisely defined uniform size and shape are generated, thereby meeting a primary prerequisite for targeting of particles to specific regions of the airway. More conventional approaches currently in use for particle generation, such as spray drying, do not produce microparticles of uniform size and shape, and as such are inferior to the methods of the present invention.

In one embodiment, the microparticles of the present invention are manufactured from a biodegradable or biocompatible polymer matrix such as a modified polylactide (poly (D, L-lactic-co-glycolic acid) (PLGA)) or polyphosphazene. The polymer matrix is designed to contain a therapeutic material (e.g. small molecular weight drug, enzymes, hormones, proteins, antibodies, vitamins, peptides, polypeptides, nucleic acids, oligonucleotides, vaccines, cells, antigens, allergens, and viruses).

In another embodiment, a degradation controlling material is added to the polymer matrix to enhance the degradation of the polymer matrix and facilitate controlled release of the therapeutic material. U.S. patent application Ser. No. 09/575,089 discloses materials and methods for the controlled release of materials from polymer matrices, and is hereby incorporated by reference in its entirety. Preferred degradation controlling materials are polyacrylic acid, polystyrene sulfonic acid, polyphosphazene, poly-L-lysine, polyaspartic acid, polymethacrylic acid, imidazole, polyglutamic acid, glycine, polystyrene maleic anhydride copolymers, polyvinylamine, polyamino acids, polyvinylpyrrolidone, vinylether maleic anhydride copolymers, and styrene-acrylic acid copolymers.

Preferably, active ingredients are incorporated into the polymer matrix either during the formation of the matrix as described in U.S. patent application Ser. No. 09/575,089, or if a porous particle is generated, introduced after the particle is formed. Additionally, small reservoirs can be fabricated into the microparticles of the present invention.

In another embodiment of the present invention, the microfabrication technologies discussed above are utilized to produce shaped microparticles containing multiple layers. In this embodiment, a drug-containing polymer layer is sandwiched between two other polymer layers that control the release of the drug. In a preferred method, a biodegradable polymer (e.g., poly(lactic-co-glycolic acid, i.e., PLGA) in an organic solvent (e.g., methylene chloride) is dried in a micro-mold, or cast as a sheet. A second layer of polymer that contains a drug, or a layer of pure drug, is then added to the mold, or cast on top of the sheet. Finally, a top layer of polymer is added to the top of the drug containing layer to form a laminar system. In the case of the mold, the layered particle is ejected from the mold. In the case of the sheets, a micro-tool is used to stamp out laminar particles.

In many therapeutic applications, protein dose delivered to the patient must be maximized. If the protein is prepared by lyophilization, however, a fluffy low density solid is formed. In another embodiment of the present invention, compressed protein microparticles are formed by first lyophilizing a protein solution in a micro-mold, and then compressing the fluffy solid with a micro-tool that fits the mold. The protein particle can be produced as part of a laminar system. The protein, which will typically be at a concentration of between 2% and 10% prior to lyophilization, is lyophilized in a micro-mold, forming a fluffy powder. The fluffy powder is then compressed with a micro-tool prior to use or further coating. Presumably, the protein can be sandwiched between two polymer layers. The first polymer layer is dried in the mold prior to addition of protein and the second layer is added after the protein is compressed.

The microparticles of the present invention can be aerosolized using dry powder inhaler systems, liquid nebulizers, or any other suitable aerosolization device.

The shaped, particulate dry powder compositions of the invention are useful for preparing aerosols for the delivery of therapeutic agents such as proteins to the respiratory tract. The term “respiratory tract” includes the upper airways, including the oropharynx and larynx, followed by the lower airways, which include the trachea followed by bifurcations into the bronchi and bronchioli. The upper and lower airways are called the conductive airways. The terminal bronchioli then divide into respiratory bronchioli, which then lead to the ultimate respiratory zone, the alveoli, or deep lung. Gonda, I. “Aerosols for delivery of therapeutic and diagnostic agents to the respiratory tract,” in Critical Reviews in Therapeutic Drug Carrier Systems, 6: 273-313, (1990). Usually, the deep lung, or alveoli, is the primary target of inhaled therapeutic aerosols for systemic delivery.

The term “biologically active agent” includes small molecules, proteins and peptides that are used for diagnostic and reagent purposes as well as small molecules, proteins and peptides that are administered to patient as the active drug substance for treatment of a disease or condition. Contemplated for use in the compositions of the invention are proteins and polypeptides such as enzymes, e.g., ascorbate oxidase, peroxidase, catalase, glucose oxidase, chymotripsin, lactate dehydrogenase and glucose-6-phosphate dehydrogenase; antibodies, e.g. Herceptin® (trastuzumab), Orthoclone OKT®3 (muromonab-CD3); hormones, e.g., insulin and human growth hormone (HGH); growth factors, e.g., fibroblast growth factor (FGF), nerve growth factor (NGF), human growth hormone releasing factor (HGHRF), and cytokines, e.g., leukemia inhibitory factor (LIF), granulocyte-colony stimulating factor (G-CSF), granulocyte macrophage-colony stimulating factor (GM-CSF), interleukin-6 (IL-6), interleukin-11 (IL-11), interleukin-9 (IL-9), oncostatin-M (OSM), and Factor VIII.

The term “biologically active” includes agents that are administered to a patient in a “therapeutically effective amount” to treat a disease or condition. As would be recognized by one skilled in the art, by “therapeutically effective amount” is meant an amount of a biologically active agent having a therapeutically relevant effect on the disease or condition to be treated. A therapeutically relevant effect relieves to some extent one or more symptoms of the disease or condition in a patient or returns to normal either partially or completely one or more physiological or biochemical parameters associated with or causative of the disease or condition. Specific details of the dosage of a particular active drug may be found in its labeling, i.e., the package insert (see 21 CFR §201.56 & 201.57) approved by the United States Food and Drug Administration.

As would be recognized by the skilled artisan, the shaped, particulate dry powder compositions of the invention may optionally include “minor amounts”, that is from about 0.05% to about 5.0% W/V and preferably from about 0.05% to from about 1.0% of a pharmaceutically acceptable excipient. Pharmaceutically acceptable excipients are those recognized by the FDA as being safe for use in humans. Additives such as, surfactants, e.g., ethoxylated dodecyl alcohol, antioxidants, e.g., Vitamin E and ascorbic acid, antimicrobials, e.g., parabens and suspending agents, e.g., povidone are contemplated for use herein.

While the selection of any particular excipient is within the skill of the art, as will be appreciated, the decision regarding whether to add an excipient and if so which one, will be made taking into account the purpose of the excipient in a specific shaped, particulate dry powder composition of the invention and if the excipient is added during the preparation of the active agent/polymer mix or after the shaped particles are formed.

In order to be pharmaceutically acceptable any formulation excipient used in a shaped, particulate dry powder composition of the invention should be recognized by the FDA as safe for use in humans. Additionally, an excipient should have no effect or minimal effect on the stability of the active agent in the compositions of the invention or on the sprayability of the shaped, particulate dry powder compositions using an electrostatic spraying means.

While the above description contains many specificities, these should not be construed as limitations on the scope of the invention, but rather as exemplification of preferred embodiments. Numerous other variations of the present invention are possible, and it is not intended herein to mention all of the possible equivalent forms or ramifications of this invention. Various changes may be made to the present invention without departing from the scope of the invention.

What is claimed:

1. A microparticle for use in the pulmonary delivery of a therapeutic material, comprising a polymer matrix, wherein said polymer matrix is prefabricated in a particular geometric shape.

2. The microparticle of claim 1, wherein said microparticle has a geometric diameter or width of about 1 to 100 microns and a thickness of about 1 to 10 microns.

3. The microparticle of claim 1, wherein said geometric shape is that of a disc, cube, rectangle, or snowflake.

4. The microparticle of claim 1, wherein said therapeutic material is a drug.

5. The microparticle of claim 1, wherein said therapeutic material is a biologically active material selected from the group consisting of enzymes, hormones, proteins, antibodies, vitamins, peptides, polypeptides, nucleic acids, oligonucleotides, vaccines, cells, antigens, allergens, viruses, and combinations thereof.

6. The microparticle of claim 1, wherein said therapeutic material is bound to, incorporated in, or encapsulated by said polymer matrix.

7. The microparticle of claim 1, wherein said polymer matrix comprises at least one biodegradable or biocompatible polymer.

8. The microparticle of claim 7, wherein said at least one biodegradable or biocompatible polymer is polylactide or polyphosphazene.

9. The microparticle of claim 1, wherein said polymer matrix further comprises at least one additional polymer for enhancing the degradation characteristics of said polymer matrix, wherein said at least one additional polymer is selected from the group consisting of polyacrylic acid, polystyrene sulfonic acid, polyphosphazene, poly-L-lysine, polyaspartic acid, polymethacrylic acid, imidazole, polyglutamic acid, glycine, polystyrene maleic anhydride copolymers, polyvinylamine, polyamino acids, polyvinylpyrrolidone, vinylether maleic anhydride copolymers, styrene-acrylic acid copolymers, and combination thereof.

10. The microparticle of claim 1, wherein said microparticle is aerosolizable by dry powder nebulizers, liquid nebulizers, and electrostatic sprayers.

11. A method for making a shaped microparticle for use in the pulmonary delivery of a therapeutic material, comprising the steps of:
(a) selecting at least one polymer or other material to form said shaped microparticle; and
(b) employing a microfabrication technique to form said shaped microparticles, wherein said technique consists of cutting said microparticles from sheets of polymer by photolithography, microstamping said microparticles from sheets of polymer, or casting said microparticles in molds.

12. A method for making a microparticle for use in the pulmonary delivery of a drug, comprising the step of sandwiching a drug-containing polymer layer between two other polymer layers.

13. A method for making a microparticle for use in the pulmonary delivery of a drug, comprising the steps of:
(a) drying a biodegradable polymer which has been dissolved in an organic solvent in a micro-mold, or casting said biodegradable polymer as a sheet;
(b) adding a second layer of biodegradable polymer which contains said drug to said micromolded or cast biodegradable polymer layer; and
(c) adding a third biodegradable polymer layer to the top of said drug containing polymer layer, whereby a laminar system is formed.

14. The method of claim 13, wherein said biodegradable polymer is poly(lactic-coglycolic acid, and said organic solvent is methylene chloride.

15. A method for making a microparticle for use in the pulmonary delivery of a protein, comprising the steps of:
(a) lyophilizing a protein solution in a micro-mold;
(b) compressing said lyophilized protein with a micro-tool compatible with said micro-mold; and
(c) sandwiching said lyophilized protein between a first polymer layer and a second polymer layer.

16. The method of claim 15, wherein the polymers of said first polymer layer and said second polymer layers are biodegradable polymers.

17. The method of claim 15, wherein said first polymer layer is dried in said micro-mold prior to addition said protein, and said second polymer layer is added after said protein is compressed.

18. A shaped, particulate dry powder composition suitable for aerosolization and delivery to the pulmonary system of a patient in need of treatment comprising a therapeutically effective amount of a biologically active agent and at least one physiologically acceptable polymer wherein said biologically active agent is contained in said polymer.

19. A composition according to claim 18 wherein the shape of said shaped particles is selected from a disc, a cube, a rectangle and a snowflake.

20. A composition according to claim 19 wherein the diameter of said shaped particles is from about 1.0 to from about 100.0 μ and wherein the shaped particle is from about 0.5 μ to about 1.5 μ thick.

21. A composition according to claim 18 wherein the biologically active agent is a protein, polypeptide or peptide.

22. A composition according to claim 21 wherein said biologically active protein, peptide or polypeptide is an enzyme, hormone, growth factor, antibody, or cytokine.

23. A composition according to claim 22 wherein said biologically active agent is selected from the group consisting of ascorbate oxidase, peroxidase, catalase, glucose oxidase, chymotripsin, lactate dehydrogenase, glucose-6-phosphate dehydrogenase, trastuzumab, muromonab-CD3, insulin, human growth hormone (HGH), fibroblast growth factor (FGF), nerve growth factor (NGF), human growth hormone releasing factor (HGHRF), leukemia inhibitory factor (LIF), granulocyte-colony stimulating factor (G-CSF), granulocyte macrophage-colony stimulating factor (GM-CSF), interleukin-6 (IL-6), interleukin-11 (IL-11), interleukin-9 (IL-9), oncostatin-M (OSM), and Factor VIII.

24. A composition according to claim 18 wherein said polymer is selected from the group consisting of PLGA, polyphosphazene, poly[(pcarboxyphenoxy)-hexane anhydride] (PCPH), polyglycolic acid (PGA), polylactic acid (PLA), polyethylene, polypropylene, poly (ethylene glycol), and poly(ethylene oxide).

25. A composition according to claim 24 wherein said polymer is PLGA.

26. A composition according to claim 18 containing from 0.1% to from about 5.0% of a pharmaceutically acceptable excipient.

27. A composition according to claim 26 wherein said excipient is selected from the group consisting essentially of surfactants, antioxidants, antimicrobials, suspending agents, and sugars.

28. A composition according to claim 18 wherein the shaped particle contains a second polymer that functions to slow the release of the active therapeutic agent from the particle.

29. A composition according to claim 28 wherein said second polymer that functions to slow the release of the active therapeutic agent from the particle.
